(19) United States
(12) Patent Application Publication
(10) Pub. No.: US 2010/0010314 A1
(43) Pub. Date: Jan. 14, 2010

(54) Endoscopic System Featuring Fiber-Pumped Fluorescent Illumination

(76) Inventors: Beat Krattiger, Beringen (CH); Martin Klumpp, Tuttlingen (DE); Manfred Kuster, Widnau (CH); Marcel Jaggli, Winterthur (CH); Sabrina Le Donne-Brechbuhl, Hallau (CH); Fritz Hensler, Neuhausen ob Eck (DE)

Correspondence Address: ST. ONGE STEWARD JOHNSTON & REENS, LLC, 986 BEDFORD STREET, STAMFORD, CT, 06905-5619, US

(21) Appl. No.: 12/465,972

(22) Filed: May 14, 2009

Related U.S. Application Data
(63) Continuation of application No. PCT/EP2007/009208, filed on Oct. 24, 2007.

(30) Foreign Application Priority Data
Nov. 14, 2006 (DE) 10 2006 053 487.5

(51) Int. Cl. A61B 1/07

(52) U.S. Cl. 600/182

(57) ABSTRACT

Disclosed is an endoscopic system comprising an excitation beam source located in a proximal supply unit, an optical radiation transmission path in an insertion piece, and a fluorescence converter at the distal end. A laser diode that emits in the shortwave visible spectral range is used as an excitation beam source while a glass fiber is used as an optical transmission path. The fluorescence converter is suitable for converting into white light and is embodied as a fluorescent element that is mounted downstream of the light emergence surface of the glass fiber as a separate, interchangeable part. Said endoscopic system is characterized in that the distal end of the glass fiber and the fluorescent element are inserted into a lighting fixture which has a light emergence opening that widens in a funnel-shaped manner. Alternatively, the fluorescent element is disposed in a replaceable head which can be coupled to the insertion piece and encompasses additional optical and heat-dissipating components in order to generate an illumination beam cluster and/or measurement beam cluster.

### CROSS-REFERENCE TO RELATED APPLICATIONS

The present application is a continuation of pending International patent application PCT/EP2007/009208 filed on Oct. 24, 2007 which designates the United States and claims priority from German patent application 10 2006 053 487.5 filed on Nov. 14, 2006.

### FIELD OF THE INVENTION

The invention relates to an endoscopic system having an excitation beam source, an optical radiation transmission path in an insertion piece, and a fluorescence converter at the distal end, where a laser diode that emits in the shortwave visible spectral range is present as an excitation beam source and a glass fiber is present as an optical transmission path and the fluorescence converter is suitable for converting into white light and where a fluorescent element acting as fluorescence converter is mounted downstream of the light emergence surface of the glass fiber as a separate, interchangeable part.

### BACKGROUND OF THE INVENTION

Application JP 2002-148 442 A discloses an illumination apparatus in which the light of a semiconductor laser is radiated into an optical glass fiber. The glass fiber consists of a light-conducting core with high refraction index, a casing with low refraction index, and a protective layer. Fluorescent dyes are embedded in the protective layer. The semiconductor laser emits in the 380-460 nm spectral range.

Part of the light is decoupled into the protective layer because of unsteadiness and impurities in the core and/or in the core-casing interface. The impurities can be imposed from outside at a defined point. The decoupling can also result from bending of the glass fiber. The fluorescent dyes in the protective layer transform blue light of the semiconductor laser into yellow light. Another part of the decoupled blue light penetrates the protective layer and attaches itself to the yellow portion to form white light. The white light is emitted over the entire length of the glass fiber that is provided with the protective layer and on which the decoupling impurities are present. The apparatus is intended essentially for the illumination in display signs or for ornamental displays.

JP 2005-205 195 A discloses an elaboration of the principle of white light generation by additive color mixing of blue laser light and in yellow light portions generated in a fluorescence converter. Light radiated by an LED or a laser diode (LD) in the blue spectral range is fed into a thin multimode glass fiber by a condenser device. The other end of the glass fiber is equipped with a wavelength converter element. Said element consists of the core of the glass fiber and a fluorescent material that surrounds the tip of the glass fiber. Because of generated white light concentrated at the tip of the glass fiber, the embodiment is particularly suitable for endoscopic applications. A number of color gradations in the fluorescent conversion and color mixing are possible thanks to the selection of laser emission wavelengths and the composition of the fluorescent material.

An optical apparatus with white light generation at the distal end of the glass fiber was presented at the trade fair “Laser 2005” in Munich, Germany, by the Nichia Corporation. A blue laser diode feeds short-wave bluish light with a wavelength of 405 or 445 nm into a thin multimode glass fiber. Situated at its end is a fluorescence converter that allows part of the blue light to pass and distributes it in diffused state. The other part of the blue light is converted into yellowish light by the fluorescent dye and is likewise radiated in diffused state. Thus, together with the directly passed-through blue light portion, a white light in turn is generated. In the process, particular emphasis was placed on exact matching of the dye and the scattering, so that the light has the most neutral possible impact.

Because of the coating of the fiber end by the fluorescence converter, the light is radiated within an angle of nearly 360 degrees. The glass fiber can be introduced with the coated head portion as illumination in the hollow space as long as the heat arising in the course of fluorescence conversion can be radiated into the hollow area without damage.

An adapter for endoscopes is disclosed in JP 2005-328 921 A, into which a fluorescent element is inserted. The adapter can be mounted on the distal end of the endoscope in such a way that the fluorescent element is situated opposite the emergence surface of an illumination fiber. By appropriate shaping of the fluorescent element and coating of its outer surface, it can be ensured that the excitation light can enter the fluorescent element and that the fluorescent light is reflected in the direction of the front surface of the fluorescent element. The front surface can be equipped with a transparent protective layer.

More recent laser light sources are on the market with progressively greater capacity output. This results in an increase in heat emission affecting the fluorescence converter, which reduces its lifetime. Thermal resistivity of the fluorescence converter can be increased by converting from organic to inorganic fluorescent components. This leads in turn, with high light emission, to a still greater heat radiation.

The concept of this white light generation by mixing a residue of blue excitation light with the fluorescent light is similar to the concept of the similarly known white light LED. With these LEDs the fluorescent dyes are applied directly on the blue-lighting LED chip. Unfortunately these white light LEDs have the great disadvantage that they possess at present only about a one- to three-fold degree of effectiveness of electrical energy (watts) to radiated light (lumens) such as halogen lamps. Therefore they also develop a great deal of heat emission, making them unsuited for endoscopic applications on the distal end. Because heat dissipation is poor as a rule on the distal end, no great heat should be generated there by illumination because it constitutes a risk of damage. This is especially important with videoscopes because their distal temperature-sensitive cameras already generate a certain heat in their own right.

It is the object of the invention to make a useful application of the principle of known white light generation in endoscopic systems for illumination and measurement beam clusters with light radiation that is essentially directed forward or selectively laterally, and to avoid heat emission affecting the fluorescence converter, the object being examined, and/or the endoscopic examination systems deployed in the vicinity of the distal illumination lens.

The object is fulfilled according to the invention through an endoscopic system of the aforementioned type owing to the decisive characteristics of claim 1 including an excitation beam source located in a proximal supply unit, an optical radiation transmission path in an insertion piece, and a fluorescence converter at the distal end, where a laser diode that emits in the shortwave visible spectral range is present as an excitation beam source and a glass fiber is present as an optical transmission path and the fluorescence converter is suitable for converting into white light and where a fluorescent element acting as fluorescence converter is mounted downstream of the light emergence surface of the glass fiber as a separate, interchangeable part, characterized in that the digital end of the glass fiber and the fluorescent element are inserted in a lighting fixture having a light emergence opening that widens in a funnel-shaped manner.

The object is also fulfilled according to the invention through an endoscopic system of the aforementioned type owing to the decisive characteristics of Claim 13 including an excitation beam source, an optical radiation transmission path in an insertion piece, and a distal-end fluorescence converter, where a laser diode emitting in the short-wave visible spectral range is present as excitation beam source and a glass fiber is present as optical transmission path and the fluorescence converter is appropriate for conversion in white light, and in which a fluorescent element is positioned downstream from the light emergence surface of the glass fiber as a separate, replaceable component, characterized in that the fluorescent element is positioned in a replaceable head that can be coupled to the insertion piece, which head is configured for generating a lighting and/or measuring beam cluster with further optical and heat-dissipating components. Advantageous elaborations are derived from the characteristics of the respective subsidiary claims.

The arrangement of a fluorescent element that is set apart and separate from the glass fiber, and thus replaceable with it, opens up diverse possibilities for geometric shaping to adapt to the specific requirements of an endoscope. The optical characteristics of the fluorescent element can likewise be extensively varied by the choice of material and the material composition. In addition, the interchangeability and installation of system units can be significantly facilitated.

In addition to the light source for white light radiation, the miniaturization of light reflectors and the beam-shaping lens assume special significance in endoscopy. If an efficient beam shaping is required, optical-geometric considerations require the fluorescent element to be as small as possible in comparison with the reflector or beam-shaping lens. This miniaturization, however, inevitably increases the heat concentration and the destructive temperature gradients. For these reasons the reduction of heat resistance in and around the fluorescent element is important. The subsidiary claims cite concepts for achieving this with miniaturized fluorescent bodies.

The term “fluorescent element” is intended to include a characteristic as diffuser for diffusing the excitation light that is let through. The diffusion is effected by diffusion centers embedded in the volume of the fluorescent element and by structural effects on the surface. Here the diffusion centers can simultaneously also be the fluorophores. Because of their dimensions, the diffusion centers can act selectively, preferably diffusing the short wavelengths.

Embodiments of the inventive system are shown schematically in the illustrations and explained more closely with reference to the illustrations.

Embodiments of the inventive system are shown schematically in the illustrations and explained more closely with reference to the illustrations.

### BRIEF DESCRIPTION OF THE DRAWINGS

FIG. 1 shows an endoscopic system with lighting fixture.

FIG. 2 shows a lighting fixture with glass fiber and fluorescent element.

FIG. 3 shows the lighting fixture in addition with crystal window.

FIG. 4 shows a replaceable head with quasi-dot-shaped fluorescent elements.

FIG. 5 shows the replaceable head from FIG. 4 with focused excitation beam cluster.

FIG. 6 shows the replaceable head from FIG. 4 with collimated excitation beam cluster.

FIG. 7a shows a larger fluorescent element in a replaceable head with lateral-directed illumination and observation.

FIG. 7b shows the same apparatus with forward-directed illumination and observation.

FIG. 8 shows the replaceable head from FIG. 7a, in addition with parallel measurement beam clusters.

FIG. 9a shows the replaceable head from FIG. 7a, in addition with generated measurement pattern.

FIG. 9b shows the same apparatus but also with video camera and electrical contacts.

### DETAILED DESCRIPTION OF THE INVENTION

The first illustration, FIG. 1, shows an endoscopic system 1 with eyepiece 2 and an insertion piece 3. The insertion part 3 can be configured as a rigid or a flexible tube. Downstream from, or in place of, the eyepiece with optical transmission of the observed image, a video camera can also be provided with display of the observed image. An excitation beam source 5 is positioned in a supply unit 4 and contains a laser diode 6 and a coupling lens 7 for feeding the excitation light into a glass fiber 8. It is also possible, of course, to provide further laser diodes with the emission of additional wavelengths whose radiation can likewise be fed into the glass fiber 8 or into additional glass fibers. This makes it possible, for instance, to compensate for spectral weaknesses of the white light. The laser diodes can be battery operated or can be supplied with energy by a network part.

To connect the supply unit 4 with the endoscopic system 1, a light conductor cable 9 is provided that is connected to the endoscope and to the supply unit 4 by special or conventional commercial plug-in connectors. Said plug-in connectors can in particular be produced so that they are autoclavable and laser-protected. The glass fiber 8 is conducted to the distal end in customary manner, loose or in a separate illumination channel or in a protective casing, through the insertion piece 3. Positioned on the distal end is an lighting fixture 10 in which the conversion into white light takes place, as well as the beam formation for illuminating the object space or for projecting a measurement beam. The lighting fixture 10 is functionally replaceable or integrated into a replaceable replacement head at the distal end of the insertion piece 3. The imaging lens is not shown here in any further detail.

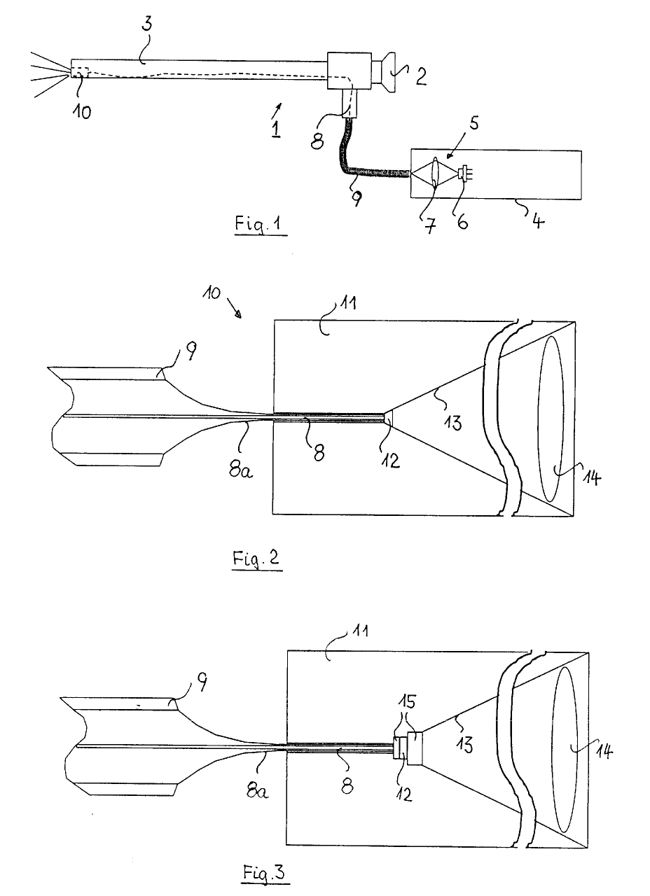

FIG. 2 shows a variant of the lighting fixture 10 in detail. The glass fiber 8 and a fluorescent element 12 are inserted in a mount 11. The mount 11 is of metal construction, for instance, such as silver, copper, or aluminum and can effectively dissipate heat that arises in the fluorescent element 12. The cross-section of the unprocessed glass fibers, consisting of sheath, casing, and core, measures about 80-900 micrometers and approximately 5-900 micrometers at the distal end 8a, which is thinned out as necessary and inserted into the mount 11. Thinning improves heat dissipation in the proximal direction. The glass fiber can also be inserted in its full cross-section into the mount 11. The light emergence opening 13 of the mount 11 widens like a funnel, for instance conically from proximal to the distal end. Inserted in the conical part of the light emergence opening 13 is a beam-shaped optical element 14.

Various parameters need to be observed in constructing the lighting fixture 10. It is generally widely known in illumination optics that the ratio of the lens diameter (reflector, lens, dissipation disk) to the source diameter (coiled filament, light arc, LED chip, fiber ends) determines the possibility of beam formation. With a point source emitter in proportion to the lens, nearly any intensity distribution can be obtained. The light outlet surface of the distal end of the glass fiber 8 is nearly point-shaped in this sense. The white light source, however, is formed by the fluorescent element 12. Its smallest possible size depends in principle on at least four attributes of the fluorescent material, namely, the temperature resistance, the heat conductivity, the light resistance, and the optical density. All four of these material attributes should be as strong as possible. To be able to construct the fluorescent element 12 as point-shaped as possible, an efficient heat dissipation must be provided. Optimally, therefore, a glass-type or transparently ceramic fluorescent element 12 is selected which consists only of inorganic parts for reasons of temperature resistance. The inorganic fluorophores bound in the fluorescent element 12 must be light resistant so that they can also convert high radiated light intensities without being damaged. The fluorophores and their concentration should be selected so that no saturation, or only a small amount, occurs through quenching. To improve heat dissipation in the proximal direction, the glass fiber diameter should be restricted to the optically necessary minimum by means of processing, which is depicted through thinning.

The light color and light distribution arise directly in and close to the fluorescent element 12 in the illustration construction, but completely within the lighting fixture 10. This makes possible a modularity in the construction of the endoscopic system 1, in that the appropriate illumination body 10, consisting of fluorescent element 12 with mount 11 and beam-shaping lens 14, can be selected during installation to suit the objective.

In the embodiment of the lighting fixture 10 according to FIG. 3, the fluorescent element 12 is inserted between two transparent disks 15 of an effective heat-conducting material, for instance a crystal or a transparent ceramic. For this purpose, sapphire or diamond is preferably chosen, so that the fluorescent element 12 can efficiently dissipate its heat on all sides. It is especially advantageous for heat dissipation if the fluorescent element 12 is also made of transparent ceramic imbued with sapphires or diamonds, because in that case the heat source and heat conductor coincide to a great degree. It is also possible here to dispense with one or both of the heat-dissipating disks 15. The heat-dissipating disks 15 can also, in addition, possess optically imaging, dispersing, reflecting, or bending properties.

The mount 11 of the lighting fixture 10 can also advantageously be constructed of a special aluminum alloy such as pure aluminum, which makes it possible in simple manner to make the surface of the conical light emergence opening 13 highly reflective. If the mount 11 is made, for instance, of copper, the conical light emergence opening 13 can also be silver-plated or plated in aluminum. The lens system 14 (lens array, prism array, diffusion panel, diffractive optical element, aspherical lens, etc.), inserted if necessary in the light emergence opening 13, forms the illumination beam, for instance round or quadrilateral, and adjusts the illumination beam to an observation objective, not illustrated here. Essential to this is the hollow conical angle of the mount 11. Also important is the hollow cone, in particular in the immediate vicinity of the fluorescent element 12. From a distance of approximately 2-10× to the diameter of the fluorescent element 12, the conical shape and the resulting direction of reflection can be dispensed with. In addition to the illustrated cone, other curved shapes are possible, including parabolas, ellipses, hyperbolas, and the like. Such forms are generally designated as funnel-shaped.

The fluorescent element 12 is shown in FIGS. 2 and 3 as a component with trapezoidal or rectilinear longitudinal section and is inserted into a correspondingly shaped recess in the conical part of the light emergence opening 13 of the mount 11. To affix it in place, the casing surface of the fluorescent element 12 can be provided with a solderable metallic layer, for instance of nickel, gold, titanium, or silver. This allows a firm soldered connection with good heat transmission to the mount 11. In the case of non-solderable aluminum as reinforcing material, cementing can also be used. The affixing of the fluorescent element 12 can also be done, of course, by clamping, which facilitates replacement.

Because the fluorescent light generated inside the fluorescent element 12 is radiated in all directions, it is advantageous to adapt the casing surface to the conical shape of the light emergence opening 13 and to make it reflective before insertion. This supports a forward direction of the radiation from the fluorescent element 12 and avoids light losses through reverse diffusion.

To adjust the color spectrum of the lighting fixture 10, the fluorescent element 12 can also be constructed of several cascading layers, which contain diverse fluorescent dyes. The color spectrum can be affected by varying the particular layers' thickness. The layer thickness can advantageously be modularly composed in simple manner of a number of fairly thin panels. This allows the color spectrum to be quickly and easily conformed to a standard during installation. This is particularly helpful when construction of the fluorescent element 12 or of fluorescent panels cannot be reproduced and is subject to fluctuations in the spectrum.

The concept of the quasi-point-shaped light source can also be realized with a replaceable head 16 coupled onto the distal end of the insertion tube 3.

FIG. 4 shows an embodiment in which a small, quasi-dot-shaped fluorescent element 12 is positioned on an efficiently heat-dissipating window 15, for instance of a transparent ceramic, sapphire, or diamond panel. The replaceable head 16 is directed in the direction of the arrow to the distal end of the insertion piece 3, so that the light outlet surface of the glass fiber 8 is positioned immediately facing the fluorescent element 12. This arrangement requires a high degree of precision in positioning. The components—window 15, fluorescent element 12, and lens system 14—can also be combined, as described above, in an lighting fixture 10 and inserted as a unit into the replaceable head 16.

Placed downstream from the lens system 14 are a deflection prism 17 and an illumination objective 18, which generates an illuminating ray cone 19 deflected by 90 degrees. Shown with broken lines in the illustration are the usual components for video recording of the illuminated object.

In the embodiment shown in FIG. 5, an imaging lens 20 is mounted upstream on the distal end of the insertion piece 3 of the light outlet surface of the glass fiber 8, to focus the exiting bundle of excitation beams when the replaceable head is in place in the fluorescent element 12. The fluorescent element 12 is positioned here between two heat-dissipating windows/panels 15. The focus of the imaging lens 20 is selected so that the excitation light is focused into the fluorescent element 12 adapted precisely to the thickness of the panel 15.

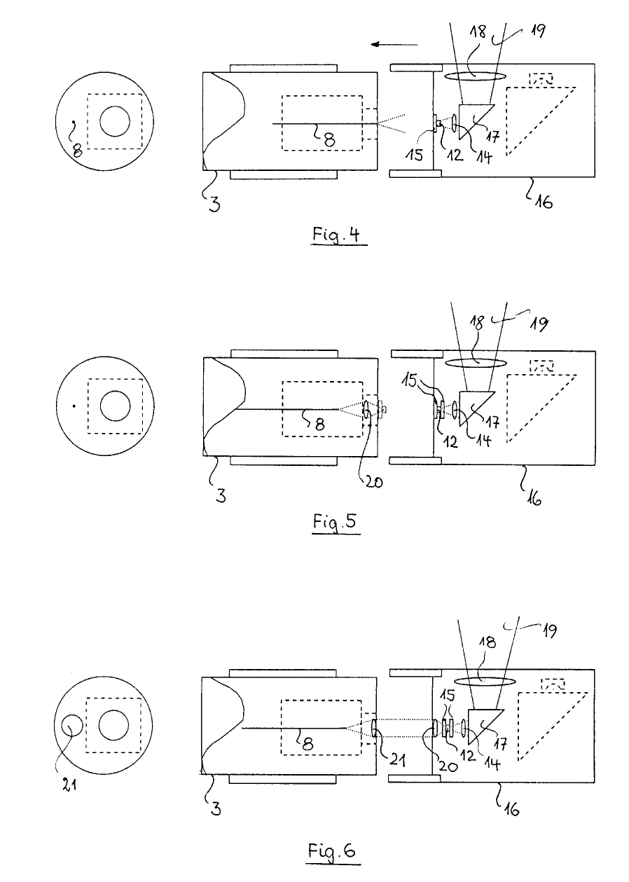

When the replaceable head's 16 position is not precisely defined, the parallel guiding of the rays is advantageous through the interface between the insertion piece 3 and the replaceable head 16, as shown in FIG. 6. Positioned on the distal end of the insertion piece 3 is a collimation lens 21 that images at infinity the excitation beam cluster issuing from the light outlet surface of the glass fiber 8. In this case the excitation light must be focused on the fluorescent element 12 with an imaging lens 20 positioned in the replaceable head 16. The variant is more complex but ensures greater tolerances in securing the replaceable head 16. With the collimated beam guidance, the greatest range of possibilities in construction are available, because the white light generation can be provided at any position in the replaceable head 16.

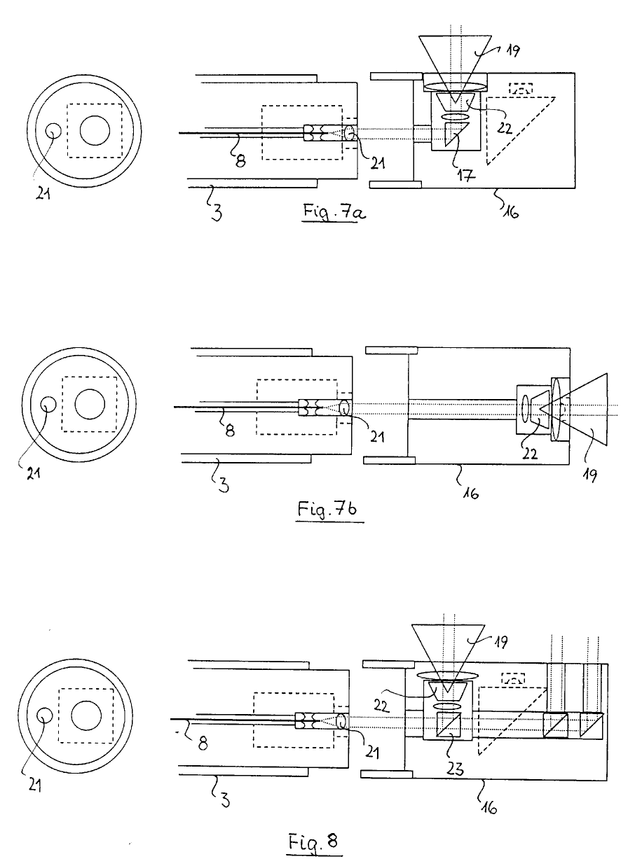

In FIG. 7a, a larger fluorescent element 22 is positioned downstream from the deflecting prism 17. The collimated excitation beam cluster is thus radiated into the fluorescent element 22. Because the radiating density is distributed over the cross-section of the beam cluster, the power density in the fluorescent element 22 is reduced. Reducing the maximum radiating density advantageously reduces fading, ageing, and heating of the fluorescent element 22. With sufficient intensity of the excitation beam cluster, part of the excitation light can still pass through the fluorescent element 22, as is indicated by the dotted continuation of the collimated excitation beam cluster through the illumination beam cone 19. Then, inside the white illumination beam cone 19, a blue spot for instance appears on the observed object and can be used as a marker. The dispersing properties of the fluorescent element 22 must be appropriately adapted to this.

FIG. 7b shows the same arrangement but with illumination and observation in forward direction.

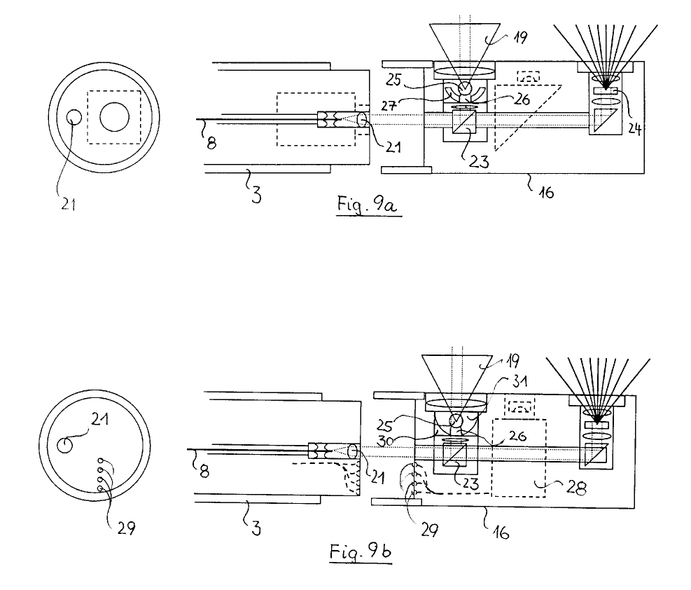

In the embodiment in FIG. 8 the collimated excitation beam cluster is split into two beam clusters by a beam splitter 23. The part reflected on the beam splitter surface is used for conversion to white light. From the portion let through, two parallel measurement beam clusters are generated, in known manner by means of optical elements that are not described in further detail, and said beam clusters constitute a comparative measurement standard for image measurement in the image. The portion of the stimulation beam cluster that is let through on the beam splitter 23 can also be used to excite an additional fluorescent element. By means of several individually excited fluorescent bodies, a shadowless illumination is realized, which improves the system protection against malfunction; alternatively, various color spectra or beaming directions can be selected.

In the embodiment seen in FIG. 9a, the collimated excitation beam cluster is likewise split. The part let through on the beam splitter 23 is divided by a diffractive optical element 24 into a number of beam clusters for generating a measurement pattern. The fluorescent element 12 in this embodiment is shown as a sphere 25 contained in a transparent, heat-conducting base 26. The base 26 and sphere 25 are surrounded by a reflector 27. The spherical shape ensures uniform radiance. Deflection of heat, however, is unfavorable because of the reduced contact surface on the base 26.

In the embodiment shown in FIG. 9b the same illuminating elements are provided as in FIG. 9a. Here, however, for observing the illuminated object area a video camera is integrated into the replaceable head 16 and is connected electrically by contacts 29 with the distal end of the insertion piece 3. The spherical fluorescent element 26 here is inserted in a reflector body 30 whose interior reflector surface, for instance of parabolic shape, is reflected. The reflector body 30 can be complemented by a transparent heat conductor 31 around the spherical fluorescent element 25.

The description of the embodiments was based on an initial assumption of the transmission of light wavelength that excited fluorescence by the glass fiber. It is also possible, however, to feed the light into the glass fiber from more than one laser diode with varying light wavelengths. Then, in the beam splitter 23 the beam-splitter surface in the replaceable head 16 must be provided with a dichroitic layer that is permeable for the wavelengths of radiance that differ from the excitation wavelength. As a result, a more favorable color for the measurement beam, for instance red or green, can be inserted to make it more recognizable.

The advantages of the fiber-pumped fluorescent illumination can be summarized as follows:
Because, in principle, just a single glass fiber is sufficient for transmitting the excitation light, better flexibility of the insertion piece is obtained with respect to curvatures in deflecting the distal end. The reduced return force of an individual fiber in relation to conventional fiber clusters leads to improvement in mechanics because the individual fiber is far more bendable than the fiber cluster.
Because of the large diameter of conventional light-conducting fiber clusters, sheering forces arise in the course of bending, both on inner and outer fibers, and said forces can tear out fibers or cause them to buckle. With individual fibers, no inner or outer tugging forces occur.
The individual fiber diameter with protective casing measures only about 80 to 900 micrometers. In comparison, the cold light cluster diameter in a conventionally illuminated video endoscope is between about 1 and 3 mm. Therefore an endoscope with individual fiber transmission of the excitation light can overall be constructed with a substantially smaller cross-section.
If one fiber is not sufficient, several fibers can radiate onto a common fluorescent element without significant increase in the cross-section, or they can each radiate their own fluorescent element. This is an easy way to optimize lighting capacity.
If the fluorescent source is small relative to the beam-forming lens, the illumination can be optimally adjusted to the field of vision.
Through the choice of fluorescent dye or dyes in the fluorescent element and/or the choice of the excitation light, the color spectrum can be adjusted. Thus, for instance, with excitation in the UV and blue in the same fiber, the spectrum can be adjusted to the optimal blackbody radiation for color reproduction. Light can also be radiated for purposes of diffusion without use of the fluorescence effect. For this purpose, various light sources, for instance, can be fed into an individual fiber with a fiber coupler. By replacing a replaceable head, it is also possible to make a spectrum change. The choice of the excitation light can even modify the color spectrum during the endoscopic examination, an advantage for instance in examinations for color modifications in the examined object.
If the fluorescent element has faded in color by ageing, it no longer emits its maximum brightness. In this case it can be replaced, for instance by exchanging a replaceable head. It is also possible to exchange only the lighting fixture or only the fluorescent element, allowing maximum reuse of parts. This reduces operating costs in comparison with permanently built-in fluorescent systems.
Because the laser diode is configured as a receptacle in the supply unit, in case of a defect it can be exchanged with the receptacle at any time. In future, if laser diodes with greater light capacity become available, the endoscopic system can be outfitted in simple manner, so that the lighting capacity at the distal end can be increased. If the higher capacity or a modified wavelength then require adjustment of the fluorescent element, that becomes possible thanks to the inventive replaceability.
Because of the insertable connection of the transmission fiber to the laser diode, and thanks to the positioning of the fluorescent body as a separate component, the fiber is replaceable at any time. This is a considerable service advantage because the fiber can be broken or torn during operation.
In using energy-efficient laser diodes for feeding the fiber, battery operation becomes possible. As a result, mobile use of the system is facilitated.
Through the use of larger lasers, light capacities up to a few watts can be transmitted to the distal end. The light quantity emitted at the distal end in this case is limited only by the fluorescent element and its thermal integration. The radiating of high intensities makes it possible to take advantage of non-linear effects.
Endoscopy is often at a disadvantage because with long, flexible endoscopes, for instance greater than 5 meters in length, the illuminating light becomes increasingly yellowish as length increases. This stems from the stronger light losses of short-wave spectral portions in light conductors. If a laser is used for excitation, on the other hand, only one wavelength is present. Consequently, no change in excitation spectrum is possible with the length, so that after conversion the radiated light retains its color, largely independently of the length of the endoscope. Minimal color modifications from non-linear conversion can be removed if necessary by capacity adjustment.
Fibers for laser transmission comprise a lower attenuation from radiation of the laser light with small numeric aperture than the white light fibers customarily used, in which the radiation of conventional illumination occurs with high numeric aperture. With the new illuminating system, therefore, considerably longer endoscopes become possible.
Conventional light sources, such as halogen lamps or gas discharge lamps, have reached the physical limits in terms of technological advances. With laser diodes or fluorescent bodies, however, we can expect new gains in capacity. The technology of the new illuminating system will therefore benefit from the continuing development of components.
The intensity of the fluorescent light is dimmable without the color essentially changing. Mechanical parts such as apertures or absorbers are not required for attenuation. Simple reduction of the excitation light causes a corresponding drop in the radiance of the converted light. Completely color-neutral dimming, on the other hand, is possible by simple pulse width modulation.
With laser diodes the intensity of the excitation light can be modified quickly and simply by modulating the laser current. By interrupting or varying the excitation light, the unconverted light can be switched off nearly instantaneously, for instance. The only other requirement is to wait for the extremely brief post-illumination of the fluorescent element. This possibility for rapid modulation is advantageous with topography measurement tasks, which require specific measurement lighting without white light illumination only for short periods.
Installation of the endoscope is simplified because no fiber trees need to be included.
The repair possibilities o the endoscope are improved because the replacement of individual fibers is simpler than exchanging a fiber cluster.
There are no longer problems with insulating porous ends of the fiber clusters against penetration by liquids.
Multi-wavelength excitation is possible with UV and blue in the same fiber in order, for instance, to adjust the spectrum better to the blackbody radiation. It becomes possible to feed into the same fiber with a fiber coupler nearly without any loss.

1. An endoscopic system comprising an excitation beam source located in a proximal supply unit, an optical radiation transmission path in an insertion piece, and a fluorescence converter at the distal end, where a laser diode that emits in the shortwave visible spectral range is present as an excitation beam source and a glass fiber is present as an optical transmission path and the fluorescence converter is suitable for converting into white light and where a fluorescent element acting as fluorescence converter is mounted downstream of the light emergence surface of the glass fiber as a separate, interchangeable part., characterized in that the digital end of the glass fiber and the fluorescent element are inserted in a lighting fixture having a light emergence opening that widens in a funnel-shaped manner.

2. The endoscopic system according to claim 1, wherein beam-shaping optical means are inserted in the funnel-shaped light emergence opening.

3. The endoscopic system according to claim 1, wherein the lighting element is of metal construction.

4. The endoscopic system according to claim 3, wherein the lighting element is made of pure aluminum, an aluminum alloy, copper, or silver.

5. The endoscopic system according to claim 3, wherein the surface of the funnel-shaped light emergence opening is configured as a mirror-like reflective surface, where the surface can also be configured as a metallic coating.

6. The endoscopic system according to claim 5, wherein the surface of the funnel-shaped light emergence opening is a mirror-like reflective surface up to a distance of 2-10× the diameter of the fluorescent element.

7. The endoscopic system according to claim 1, wherein a heat-dissipating window is positioned upstream and/or downstream from the fluorescent element in the light direction.

8. The endoscopic system according to claim 7, wherein the window is produced as a diamond panel or a transparent, diamond-layered element, which can also possess imaging, reflecting, dispersing, or bending properties.

9. The endoscopic system according to claim 1, wherein a single-mode fiber is present as glass windows.

10. The endoscopic system according to claim 1, wherein the fluorescent element consists of glass-type or ceramic material with embedded fluorophores.

11. The endoscopic system according to claim 10, wherein the fluorescent element is composed of several layers with differing fluorophores and/or concentrations of the fluorophores.

12. The endoscopic system according to claim 1, wherein the surfaces of the fluorescent element connected with the lighting element are at least partially reflecting.

13. An endoscopic system having an excitation beam source, an optical radiation transmission path in an insertion piece, and a distal-end fluorescence converter, where a laser diode emitting in the short-wave visible spectral range is present as excitation beam source and a glass fiber is present as optical transmission path and the fluorescence converter is appropriate for conversion in white light, and in which a fluorescent element is positioned downstream from the light emergence surface of the glass fibers as a separate, replaceable component, characterized in that the fluorescent element is positioned in a replaceable head that can be coupled to the insertion piece, which head is configured for generating a lighting and/or measuring beam cluster with further optical and heat-dissipating components.

14. The endoscopic system according to claim 13, wherein an imaging lens for focusing the excitation beam cluster exiting from the light emergence surface of the glass fiber onto the fluorescent element is positioned on the distal end of the insertion piece.

15. The endoscopic system according to claim 13, wherein a heat-dissipating window is positioned upstream and/or downstream from the fluorescent element in the replaceable head.

16. The endoscopic system according to claim 13, wherein the fluorescent element is inserted between two heat-dissipating windows.

17. The endoscopic system according to claim 16, wherein at least one window is constructed as a diamond panel or transparent, diamond-layered element, which can also possess optically imaging, reflecting, diffusing, or bending properties.

18. The endoscopic system according to claim 13, wherein an imaging lens is positioned on the distal end of the insertion piece for generating a collimated beam cluster from the excitation beam cluster emerging from the light emergence surface of the glass fibers.

19. The endoscopic system according to claim 18, wherein an imaging lenses for focusing the collimated excitation beam cluster onto the fluorescent element is positioned in the replaceable head.

20. The endoscopic system according to claim 18, wherein an optical element is positioned in the replaceable head for adapting the beam density of the collimated excitation beam cluster to the geometric and fluorescent-optical properties of the fluorescent element.

21. The endoscopic system according to claim 13, wherein a beam-diverging element is positioned in the replaceable head on the light emergence side.

22. The endoscopic system according to claim 21, wherein the beam-diverging element is configured as a beam splitter.

23. The endoscopic system according to claim 22, wherein the beam splitter is configured as a cube element with beam-splitting and beam-diverting cementing surface.

24. The endoscopic system according to claim 22, wherein at least one additional beam source with emission in the red or green spectral range and for coupling into the glass fiber is present in the supply unit, where the beam splitter is provided with a dichroitic layer for reflecting or transmitting the additional beam.

25. The endoscopic system according to claim 13, wherein an optical imaging means is positioned upstream from the fluorescent element on the light emergence side for generating a lighting beam cluster with predetermined opening.

26. The endoscopic system according to claim 13, wherein the fluorescent element is surrounded on all sides by a transparent heat-dissipating medium.
